(19) United States
(12) Patent Application Publication
(10) Pub. No.: US 2003/0104486 A1
(43) Pub. Date: Jun. 5, 2003

(54) Methods and apparatus for detecting and quantifying lymphocytes with optical biodiscs

(76) Inventor: Gowri Pyapali Selvan, Irvine, CA (US)

Correspondence Address: HALE AND DORR, LLP, 60 STATE STREET, BOSTON, MA, 02109

(21) Appl. No.: 09/988,728

(22) Filed: Nov. 20, 2001

Related U.S. Application Data
(60) Provisional application No. 60/249,136, filed on Nov. 16, 2000.
(60) Provisional application No. 60/259,806, filed on Jan. 4, 2001.
(60) Provisional application No. 60/302,757, filed on Jul. 3, 2001.

(51) Int. Cl. G01N 33/53; G01N 33/567; G06F 19/00; G01N 33/48; G01N 33/50

(52) U.S. Cl. 435/7.2; 702/19

(57) ABSTRACT

A clinical diagnostic assay is performed on an optical bio disc and is read with a disc drive. A method for determining the quantity of a specific type of blood cell in a biological sample includes binding an antibody to a capture zone on the disc, providing a sample to the capture zone, remove portions of the sample that are not bound in the capture zone, and counting bound cells.

### CROSS-REFERENCES TO RELATED APPLICATIONS

This application claims the benefit of the following provisional patent applications: Serial No. 60/249,136, filed Nov. 16, 2000; Serial No. 60/259,806, filed Jan. 4, 2001; and Serial No. 60/302,757, filed Jul. 3, 2001. These applications are hereby incorporated by reference into the subject application in their entireties.

### BACKGROUND OF THE INVENTION

The present invention relates to the field of diagnostic assays and biological analysis.

Blood count screening is a routine clinical test, and is frequently used to diagnose or assess conditions including acute or chronic diseases, infections, fevers, injuries, parasitic diseases, myeloid disorders, anemias, and leukemias, and during treatment with myelosuppressive drugs. Blood counting is also routine to secure a baseline before surgical procedures or blood transfusions. A complete blood count (CBC) is a collection of tests including the determination of hemoglobin, hematocrit, mean corpuscular hemoglobin, mean corpuscular hemoglobin concentration, mean corpuscular volume, platelet count, and white blood cell count. A blood count also includes an enumeration of the erythrocytes or red blood cells (RBC) and leukocytes or white blood cells (WBC) in a unit of whole blood. In a normal healthy person, the WBC counts are typically 4000 to 10800 cells per μl. Factors such as exercise, stress, and disease can affect these values. A high WBC may indicate infection, leukemia, or tissue damage, and there is increased risk of infection if the WBC falls below 1000 cells per μl.

Leukocyte differential testing is used to distinguish amongst the five major classes of leukocytes: neutrophils, lymphocytes, monocytes, eosinophils and basophils. Leukocyte differential testing is essential to gather information beyond that obtainable from the leukocyte count itself, and is used to evaluate diseases such as leukemia, eosinophilia, monocytosis, and basophilia. Repeated testing for leukocytes or leukocyte differential also may be performed in the connection with severe leukopenia (e.g., secondary to drug therapy). During chemotherapy or radiation therapy, leukocyte differential counts are important to determine if the treatment is significantly depleting healthy white blood cells in addition to cancerous cells.

Differential leukocyte counts are typically determined by computerized cell counting devices. These devices determine the total count and the percentages of the five major white cell types. In normal individuals, there are a majority of neutrophils (50-60%), followed by lymphocytes (20-40%), then monocytes (2-9%), with fewer eosinophils (1-4%) and basophils (0.5-2%).

Within the category of lymphocytes, there are further sub-types of cells. For example, lymphocytes can be broadly divided into T-cells (thymus—derived lymphocytes) and B-cells (bursal—equivalent lymphocytes), which are largely responsible for cell-mediated and humoral immunity respectively. Although morphological characteristics have been used to classify groups within the leukocytes, morphology alone has proved inadequate in distinguishing the many functional capabilities of lymphocyte sub-types. To distinguish lymphocytes with various functions, techniques including analysis by resetting, immuno-fluorescence microscopy, enzyme histochemistry, and recently, monoclonal antibodies have been developed.

T-cells are often further distinguished by the presence of one of two major cell surface antigens: CD4 and CD8. CD4+ cells are referred to as helper T-cells and are involved in antibody-mediated immunity. These T-cells bind to antigens presented by B-cells and cause the development of a clone of plasma cells which secrete antibodies against the antigenic material. CD4+ T-cells are also essential for cell-mediated immunity. CD4+ T-cells bind to antigen presented by antigen-presenting cells (APCs) such as phagocytic macrophages and dendritic cells, and release lymphokines that attract other immune system cells to the area. The result is inflammation, and the accumulation of cells and molecules that attempt to wall off and destroy the antigenic material.

CD8+ T-cells are referred to as cytotoxic or killer T-cells. These T-cells secrete molecules that destroy the cell to which they have bound. This is important in fighting viral infections, as the CD8+ T-cells destroy the infected cells before they can release a fresh crop of viruses able to infect other cells.

The estimation of CD4+ and CD8+ T-cells and the ratio of CD4+/CD8+ T-cells is useful to assess the immune health of human patients with immune-compromised diseases. For example, the human immunodeficiency virus (HIV) is a retrovirus with high affinity for the CD4 cell surface antigen, and therefore CD4+ T-cells are potent targets for the virus. Acquired immune deficiency syndrome (AIDS) provides a vivid and tragic illustration of the importance of CD4+ T-cells in immunity. As the disease progresses, the number of CD4+ T-cells declines below its normal range of about 1000 per μl, as the patient's CD8+ T-cells destroy the infected CD4+ T-cells and/or infected CD4+ cells undergo apoptosis or cell suicide. Thus, the ratio of CD4+ to CD8+ T-cells provides a diagnostic marker for the progression of the disease. The U.S. Public Health Service recommends that CD4+ levels be monitored every 3-6 months in all infected persons (40 million tests are done every year in 600 testing laboratories in the United States).

In addition to CD4 and CD8, there are many other cell surface antigens (e.g., CD3, CD16, CD19, CD45, CD56) which can be used to identify sub-types of lymphocytes. The ability to detect these cell surface antigens by antibody techniques has added a new dimension to diagnostic pathology, and a variety of techniques are available for the study of immunophenotypes of hematolymphoid disorders (e.g., AIDS, leukemias, and lymphomas). Conventional microimmunoassays such as radio-immunoassays (RIA), enzyme-immunoassay (EIA), fluorescence-immunoassay (FIA) use an isotope, an enzyme or a fluorescent substance in order to detect the presence or absence of corresponding antibodies or antigens, respectively, that react specifically therewith.

The U.S. Centers for Disease Control (CDC) have set out the following guidelines for clinicians to follow when determining white blood cell quantities: (1) preferably use blood within 30 hours, and no later than 48 hours, from the time it is drawn; (2) count a minimum of 10,000-30,000 cells; (3) identify CD4+ cells as those positive for both CD3 and CD4; (4) identify CD8+ cells as those positive for both CD3 and CD8; (5) identify CD45+ cells as lymphocytes; (6) identify CD19+ cells as B-lymphocytes; (7) identify CD16+/CD56+ cells as NK cells; and (7) run positive and negative controls. It is most often the case that whole blood is used in a multi-platform, three stage process which includes the determination of: (1) the white blood cell (WBC) count; (2) the percentage of WBC's that are lymphocytes (differential) and (3) the percentage of lymphocytes that are CD4+ T-cells (flow cytometry). These steps are often carried out in a clinical setting, such as hospital, contract laboratory, or clinic, with equipment which is expensive and difficult to obtain and operate. Serological tests are routinely performed by clinics, medical and veterinary hospitals, the Red Cross and other institutions.

### SUMMARY OF THE INVENTION

Improved means for carrying out analyses on samples, such as a subject's blood, in a timely, cost efficient and technically relevant way are needed. In particular, there is a need for easier, more efficient ways to quantify the relative levels of various types of white blood cells, or other cell types, which exist in a subject's blood or body fluids.

The embodiments of the present invention includes methods and apparatus for conducting an assay in association with an optical analysis disc to detect and count cells, and particularly lymphocytes. In one aspect, a method includes providing a sample in or on a disc surface, the disc having encoded information which is readable by an optical reader. This information can be used to control the scanning of the reader relative to the disc. The disc is loaded into the optical reader, and an incident beam of electromagnetic radiation from a radiation source is directed to the disc. The beam is scanned over the disc by rotating the disc about a central axis and by moving the incident beam in a direction radial to the axis. A beam of electromagnetic radiation either transmitted through or reflected from the disc is detected and analyzed to extract information characteristic of the sample.

The embodiments of the invention also include a disc with a substrate and cap spaced to form a chamber. A sample of material, such as blood with cells, is provided in the chamber. When the disc is rotated, the sample moves past capture zones. At the capture zones are capture layers with antibodies or other specific binding partners that bind to antigens such as CD4 and CD8. Preferably one test can be used to image CD4 and CD8 and other antigens in a blood sample. The embodiment also includes a disc reader for directing light to viewing windows where the capture zones are located, and using transmitted or reflected light to detect and count captured cells. These CD4 and CD8 counts, and the ratio between them, are useful for monitoring conditions such as AIDS.

The sample is preferably provided to the chamber within a disc. A single chamber preferable has multiple capture areas, each of which may have one or more antibody. In one embodiment, a single channel has multiple capture zones, each with a different type of antibody, and may have capture zones that serve as control zones. These capture zones can be aligned along one or more radii of the disc. Detection methods include detecting transitions in the feature, or imaging the viewing window and using image recognition software to count captured cells. Counting may be direct, such as counting a desired cell: or indirect, such as counting a collection of desired and non-desired cells, counting non-desired cells, and subtracting to obtain a count of desired cells. The capture zone may have one or more layers of antibodies.

When a sample of cells is provided to the disc, the disc can be rotated in one or more stages to move the cells to the capture zones, then to move unbound cells away from the capture zones. The sample may be processed in other ways, e.g., incubated or heated with the light source that is used for detection. Microfluidics can be used to add stain or any other liquids that may be desired for on-disc processing of the sample. This processing is preferably specified in encoded information on the disc in informational areas, which cause the drive and reader to rotate at desired speeds and for desired time, with intermediate other steps, such as incubation.

The methods and apparatus can have one or more of the following advantages: a simple and quick processing on the disc without requiring an experienced technician to run a test, small sample volume, and use of inexpensive materials and known optical disc formats and drive manufacturing. These and other features and advantages will be better understood by reference to the following detailed description when taken in conjunction with the accompanying drawings.

### BRIEF DESCRIPTION OF THE DRAWINGS

FIGS. 1A-1C are an exploded perspective view, top view, and partially cut-away perspective views of a reflective disc according to an embodiment of the present invention.

FIGS. 2A-2C are an exploded perspective view, top view, and partially cut-away perspective views of a transmissive disc according to an embodiment of the present invention.

FIG. 3 is a pictorial and schematic diagram of an optical disc reading system according to an embodiment of the present invention.

FIG. 4 is a pictorial view showing the attachment of antibodies to a white blood cell according to an embodiment of the present invention.

FIG. 5 is a pictorial diagram of an optical disc having chambers to illustrate a “bar code” technique according to an embodiment of the present invention.

FIGS. 6A-6C are cross-sectional views showing a sample introduced in a disc according to an embodiment of the present invention.

FIG. 7A is an illustration of results obtained from an assay using the “bar code” format according to an embodiment of the present invention.

FIG. 7B shows corresponding microscope and disc images for CD4, CD8, and control regions.

FIG. 8 shows a larger view of corresponding microscope and disc images.

FIGS. 9 and 10 illustrate the use of image recognition according to an embodiment of the present invention.

FIG. 11 is a screen shot of output from the bar code according to an embodiment of the present invention.

FIG. 12 shows a method for going from captured cells to usable output according to an embodiment of the present invention.

FIG. 13 shows a method for preparing samples and having an assay done on a disc according to an embodiment of the present invention.

### DETAILED DESCRIPTION

The invention relates to clinical diagnostic assays and related optical bio-discs and a disc drive system.

The invention includes a method for determining the number of blood cells with specified cell surface antigens in a biological sample, including providing blood cells in a chamber in an optical disc under binding conditions, the disc having a surface with at least one capture zone within the chamber, and wherein the capture zone has an anti-ligand, such as an antibody or aptamer, which binds to a cell surface ligand (antigen) present on the blood cell. The disc is rotated to remove unbound cells from the capture zone. The capture zones are located within viewing windows for allowing light to be directed to the captured cells. Light directed to the viewing windows is reflected or transmitted and then detected to provide a signal. A calculation can then be made using the signal to determine the quantity of the specific type of blood cell in the biological sample.

Some embodiments relate to use of a CD family of assays including a method for determining the CD4+/CD8+ cell ratio from a sample of blood from a subject and using that ratio to diagnose or monitor the progress of a condition or disease in that subject. The subject may be a mammal (e.g., a rodent, monkey, or human). The invention provides for a solid phase cell capture assay for rapid and cost efficient determination of absolute numbers of CD4+ and CD8+ T-lymphocytes and for the calculation of the ratio of CD4+/CD8+ lymphocytes in blood samples.

In some embodiments, antibodies attached to the capture field are specific for a particular cell surface antigen. These antibodies specifically bind a cell surface antigen expressed on a specific cell type, thereby allowing that cell type to become bound to the capture field under antibody binding conditions. In some embodiments, antibodies affixed to any one particular capture field are all specific for the same antigen. In other embodiments, there are different antibodies affixed to one particular capture field, thereby allowing several different types of cells to be bound by the capture agents.

One benefit of the present invention is that the cost of an analysis of a sample can be much less expensive than a test run in a standard clinical setting. For example, the methods described herein are capable of being carried out by a relatively unskilled person in almost any location. The person would only need to be able to obtain a small sample of a subject's blood or other bodily fluid. Therefore, embodiments of this invention are useful globally, including in remote villages where traditional clinically diagnostic equipment is generally not available. The blood sample is then loaded into a chamber within the disc where a capture field having capture antibodies is located. The disc is placed into a drive assembly and rotated at about 2000 rpm to 5000 rpm for about one to five minutes, or at any suitable speed and time sufficient to first cause cells to move to capture zones, and then to cause cells which are not bound by the capture antibodies to be moved from the capture zones and collected in a separate part of the chamber (e.g., in a waste receptacle in the chamber or outside of the chamber). A series of tests can be performed on one sample on a single disc, thereby increasing the cost and time efficiency of the analysis.

The embodiments of assays and methods use localized cell capture at capture zones on the optical disc. Several specific cell capture zones are created on a disc by localized application of various monoclonal or polyclonal antibodies to particular white blood cell antigens. The specifics of various chemistries encompassed by the invention are discussed more fully hereinbelow.

The biological assays are performed within an optical disc that includes a chamber having specific antibodies attached to the solid phase associated with that chamber. In one example, a method is described for the determination of the absolute numbers of cells (such as lymphocytes) expressing specific cell surface antigens (e.g., CD2, CD4, CD8, CD3, CD14, CD15, CD16, CD19, CD45 and/or CD56) captured by specific antibodies affixed to the capture zone(s). In this method, a small quantity (e.g., 7 μl) of mononuclear cells (MNC) isolated from whole blood is loaded into a chamber in an optical disc. Upon flooding the chamber with the MNC blood (e.g., approx. 30,000 cells/μl), cells expressing those cell surface antigens are captured in the capture zones on the disc via binding of the cell surface antigen to the specific antibody which was previously bound to the surface at the capture zone. An optical disc may have multiple capture zones within one chamber. A grouping of several capture zones is referred to herein as a “bar code” because the data resulting from cells binding at certain capture zones resembles the dark and light striped pattern known as a bar code. In some embodiments, the bar code incorporates defined negative control areas and positive control areas.

An optical disc drive system is employed to rotate the disc, read and process any encoded information stored on the disc, and analyze the cell capture zones in the chamber. The disc drive is provided with a motor for rotating the disc, a controller for controlling the rate of rotation of the disc, a processor for processing return signals from the disc, and an analyzer for analyzing the processed signals. The rotation rate is variable and may be closely controlled both as to speed and time of rotation. The disc may also be utilized to write information to the disc either before, during, or after the assay, such as test results to keep them with the sample. The sample in the chamber at the capture zones is interrogated by the read beam of the drive and analyzed by the analyzer. The disc may include encoded information for controlling the rotation of the disc, providing processing information specific to the type of immunotyping assay to be conducted and for displaying the results on a monitor associated with the drive.

Other examples of evaluation methods for blood samples include methods to determine the ratio of T-helper cells (inducer cells) to suppressor T-cells (cytotoxic T-lymphocytes). The methods encompass evaluation tests in CD, CD-R, DVD, or other disc formats. Variations or alternative versions thereof according to the present invention include a robust capture chemistry that is stabilized on the disc. Unbound non-specific cells are spun off, leaving behind specific target cells from the blood sample which are specifically bound at a capture zone on the disc. The read or interrogation beam of the drive detects the captured cells and generates images. The cells can then be counted using image recognition software, or by detecting transitions between light and dark in a transmitted and/or reflected beam of light.

Another example of blood cell analysis which can be performed using the present invention is an immunotyping assay. A panel of capture zones having antibodies specific for particular immunological cell surface determinants are prepared. A CD2+ and CD19+ panel of capture zones are prepared on a disc and are used to quantify the T-lymphocytes and B-lymphocytes in a blood sample. A CD4+ and CD8+ panel of capture zones is prepared on a disc and the panel is used to quantify sub-types of T-lymphocytes in a blood sample. T-lymphocytes (CD4+ and CD8+) are elements of the immune system in humans. The pathogenesis of acquired immunodeficiency syndrome (AIDS) is attributed to decreases in the number of CD4+ T-lymphocytes. The Public Health Service recommends that CD4+ levels be monitored every 3-6 months in all infected persons. Two million tests are done every year in 600 testing laboratories in the United States alone.

In an embodiment of the present invention, the capture zones of the optical disc may contain affixed thereto the following: antibodies to CD45 to determine the total number of lymphocytes; antibodies to CD2; antibodies to CD3 and CD19 to determine the total number of lymphocytes; antibodies to CD3 to determine the total number of T-lymphocytes; antibodies to CD8/CD4/CD16 to determine the total number of T-cells; and antibodies to CD4 to determine the total number of helper T-cells. From these numbers, which may be determined at the same time, clinically and diagnostically important percentages can be calculated.

Thus, the present invention includes the capture and counting of white blood cells in defined areas of an optical bio-disc using specific antibodies. The measurements are quantitative and reproducible. The total number of white blood cells can be measured. The percentage of white blood cells that are lymphocytes is another determination that can be made using embodiments of the present invention. The percentage of lymphocytes that are T-cells is a further analysis made using the present invention.

The methods of the present invention are useful for the determination of an allergic response in a subject, for evaluating and monitoring immune responses, for monitoring and evaluating the relative health or immunological status of a person infected with HIV, or for determining the relative health of the immune system of an individual. The present invention is also useful in the diagnosis of blood disorders, such as leukemia.

In carrying out the methods described herein, the volume of the sample in the chamber in the optical disc should be constant, the mixture of cells above each capture field should be representative of the whole population of white blood cells in the sample, the antibodies should be highly specific to one cell type, an accurate measurement of the efficiency of each capture zone should be possible, and the number of cells captured at each capture zone should reflect their number in the sample.

The embodiments of the present invention include methods for determining cell counts. These determinations are useful in the course of diagnosing a disease condition in a human subject or in monitoring the progression of a disease in a subject or in monitoring the effects of certain treatments on a diseased state in a human subject. Furthermore, the assays and methods and apparatus described as embodiments are useful in the identification of changes in cell surface antigens in pathological conditions. For example, in leukemia, an assay could be used to identify an increase in B-cell or T-cell antigens, such as CD2 and CD19. A decrease in CD4+ T-lymphocytes would be an indication of the progression of AIDS in a subject. In addition, the present invention is useful to monitor white blood cells in patients undergoing therapeutic treatments like radiation and chemotherapy. Cancer patients with B-cell leukemia show an increase in B-cell antigens like CD19 and this characteristic can be identified using the present methods.

The assays described herein can replace those now routinely carried out in clinical laboratories, such as hospital and service laboratories, and can make it possible for such assays to be carried out at the point of patient care, e.g., physician offices, in-patient service centers, emergency vehicles/rooms, and at-home testing. The methods of the present invention also can be used as portable testing and detection systems. The apparatus and methods described herein are useful in military and civilian defense contexts for detecting immunological reactions to the environment or to chemical or biological warfare agents. The methods and systems described herein also are useful in food testing and in water safety testing. Benefits of this technology include lower costs of both the instrumentation and necessary reagents; speed, sensitivity, reproducibility and accuracy; and the ability to carry out many assays simultaneously.

Blood cells which can be separately or collectively identified using the present methods include, but are not limited to, neutrophils, monocytes, basophils, eosinophils, granulocytes, and lymphocytes.

One embodiment of the present invention is referred to as a “bar code.” This technique allows for the qualitative and/or quantitative analysis of blood samples. One disc can have several capture zones, each of which has affixed thereto a capture antibody which is specific for a particular antigen on the surface of a white blood cell. These capture zones can be in one or more separate chambers, although a single chamber can be beneficial. Multiple chambers with identical sets of capture zones can be used to test different samples, or to provide redundancy for one sample. A test result characteristic of a particular disease state, a normal state, or some other condition can be predetermined as a readout. Therefore, the subsequent analysis of blood samples from subjects can be compared to the known bar code result in order to diagnose the condition of that subject immediately. For example, one elongated chamber on a disc can include separate capture fields which are arranged in a row and are in fluid communication with one another and which have affixed thereto capture antibodies specific for CD2 (T-cell specific), CD19 (B-cell specific), CD44/CD45 (leukocyte specific), CD4, and CD8 (lymphocyte-subsets).

Once the mononuclear cells from the blood sample are bound at the capture zones, the cells specifically attached can be imaged microscopically using a cellular dye. Dyes useful for this include LI-COR (LICOR Biosciences, Lincoln, Nebr.); TO-PRO-5-iodide (Molecular Probes, Inc., Eugene, Oreg.), IR-780 iodide (Sigma-Aldrich, St. Louis, Mo.), Streptavidin Laser Pro (Molecular Probes), or dd-007 (LICOR).

A vital stain which does not require any further processing is preferred for use in the present invention. The vital stain selected should intensely label all the different types of blood cells, in particular, the white blood cells. ZynoStain (Hema Technologies, Inc.) is one example of such a stain. In carrying out the present invention, the disc reader can distinguish different types of cells and different types of white blood cells. The stain may be provided in advance on the disc and then is moved to the capture zones through microfluidics and the rotational motion.

Optical Bio-Disc

An optical bio-disc for use with embodiments of the present invention may have any suitable shape, diameter, or thickness, but preferably is implemented on a round disc with a diameter and a thickness similar to those of a compact disc (CD), a recordable CD (CD-R), CD-RW, a digital versatile disc (DVD), DVD-R, DVD-RW, or other standard optical disc format. The disc may include encoded information, preferably in a known format, for performing, controlling, and post-processing a test or assay, such as information for controlling the rotation rate of the disc, timing for rotation, stopping and starting, delay periods, multiple rotation steps, locations of samples, position of the light source, and power of the light source. Such encoded information is referred to generally as operational information.

The disc may be reflective, transmissive, or some combination of reflective and transmissive. In the case of a reflective disc, an incident light beam is focused onto a reflective surface of the disc, reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light.

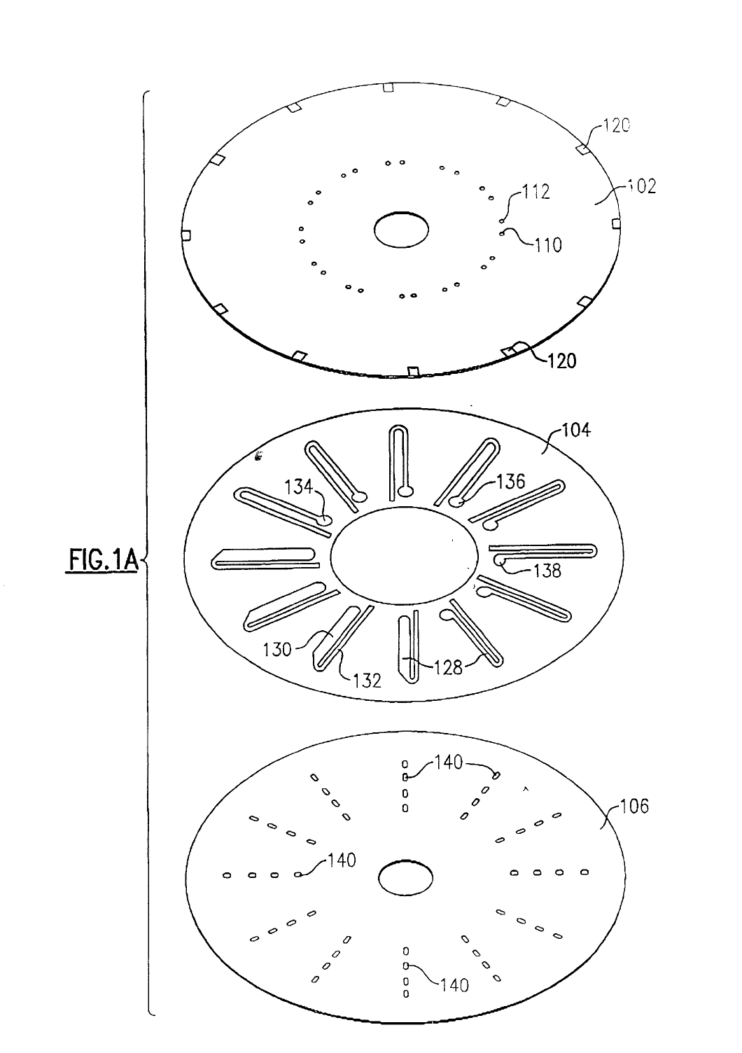

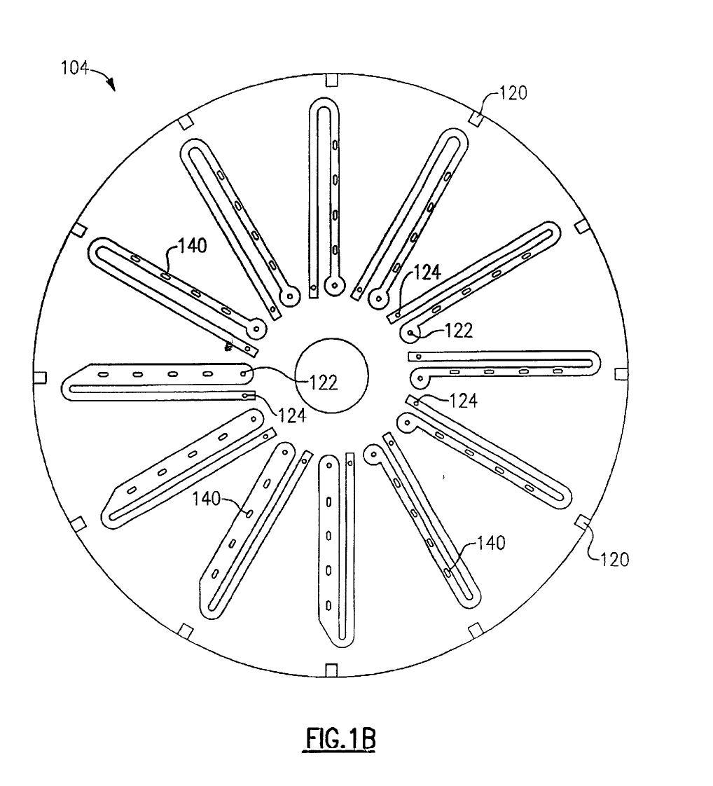

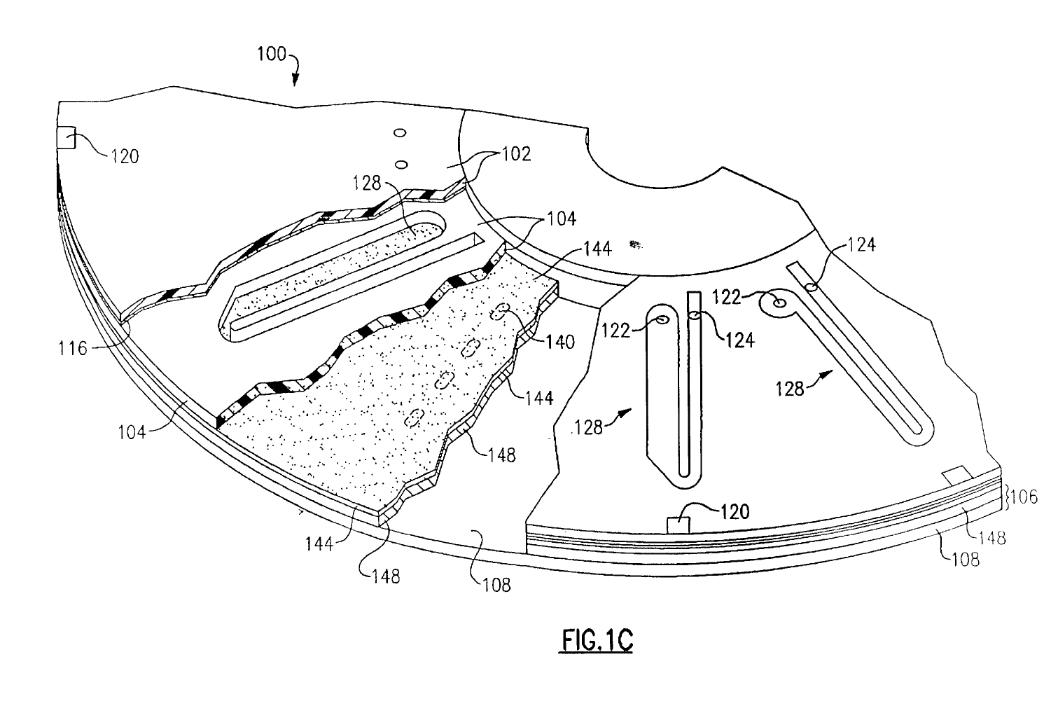

Referring to FIGS. 1A, 1B, and 1C, a reflective disc 100 is shown with a cap 102, a channel layer 104, and a substrate 106. Cap 102 has inlet ports 110 for receiving samples and vent ports 112. Cap 102 may be formed primarily from polycarbonate, and may be coated with a reflective layer 116 on the bottom thereof. Reflective layer 116 is preferably made from a metal. such as aluminum or gold.

Channel layer 104 defines fluidic circuits 128 by having desired shapes from channel layer 104. Each fluidic circuit 128 preferably has a flow channel 130 and a return vent channel 132, and some have a mixing chamber 134. A mixing chamber 136 can be symmetrically formed relative to the flow channel 130, while an off-set mixing chamber 138 is formed to one side of the flow channel 130. Fluidic circuits 128 can include other channels and chambers, such as preparatory regions or a waste region, as shown, for example, in U.S. Pat. No. 6,030,581, which is incorporated herein by reference. Channel layer 104 can include adhesives for bonding substrate to cap.

Substrate 106 has polycarbonate layer 108, and has target zones 140 formed as openings in a reflective layer 148 deposited on the top of layer 108. Target zones 140 may be formed by removing portions of reflective layer 148 in any desired shape, or by masking target zone areas before applying reflective layer 148. Reflective layer 148 is preferably formed from a metal, such as aluminum or gold, and can be configured with the rest of the substrate to encode operational information that is read with incident light, such as through a wobble groove or through an arrangement of pits. Light incident from under substrate 106 thus is reflected by layer 148, except at target zones 140, where it is reflected by layer 116. Target zones may have imaged features without capture, while a capture zone generally refers to a location where an antibody or other anti-ligand is located.

Referring particularly to FIG. 1C, optical disc 100 is cut away to illustrate a partial cross-sectional view. An active capture layer 144 is formed over reflective layer 148. Capture layer 144 may generally be formed from nitrocellulose, polystyrene, polycarbonate, gold, activated glass, modified glass, or a modified polystyrene, for example, polystyrene-co-maleic anhydride. Channel layer 104 is over capture layer 144. Polystyrene is generally preferred for a WBC capture zone.

Trigger marks 120 are preferably included on the surface of a reflective layer 148, and may include a clear window in all three layers of the disc, an opaque area, or a reflective or semi-reflective area encoded with information. These are discussed below.

In operation, samples are introduced through inlet ports 110 of cap 102. When rotated, the sample moves outwardly from inlet port 110 along capture layer 144. Through one of a number of biological or chemical reactions or processes, detectable features may be present in the target zones. These features are referred to as investigational features. Examples of such processes are shown in the incorporated U.S. Pat. No. 6,030,581.

The investigational features captured by the capture layer may be designed to be located in the focal plane coplanar with reflective layer 148, where an incident beam is typically focused in conventional readers; alternatively, the investigational features may be captured in a plane spaced from the focal plane. The former configuration is referred to as a “proximal” type disc, and the latter a “distal” type disc.

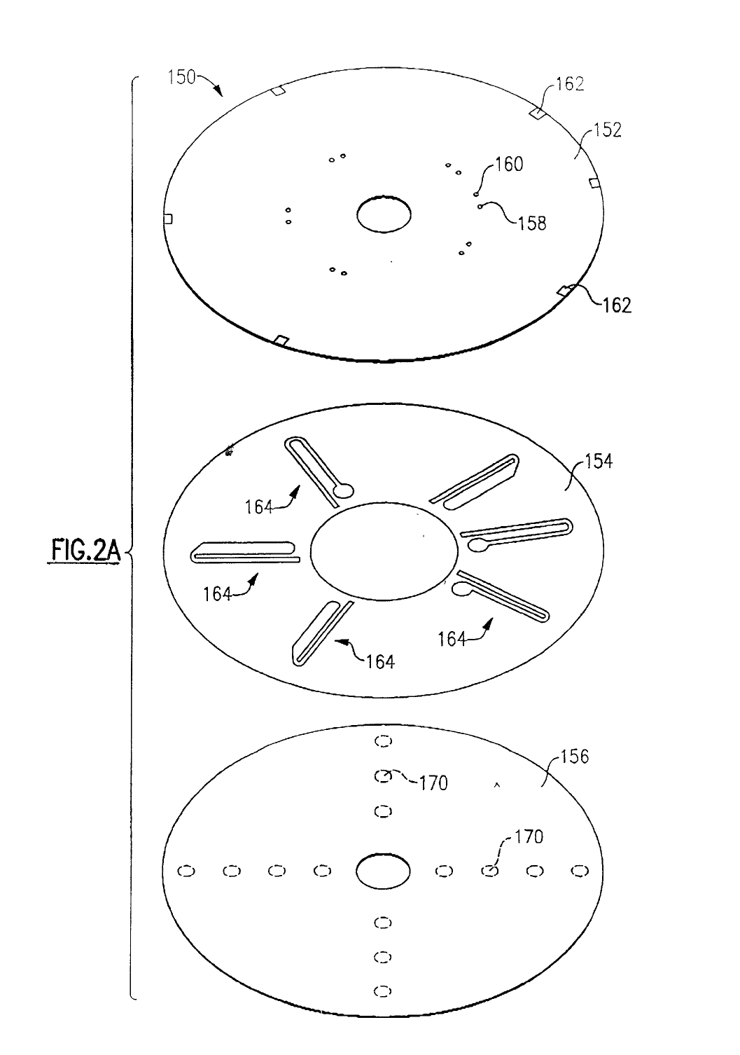

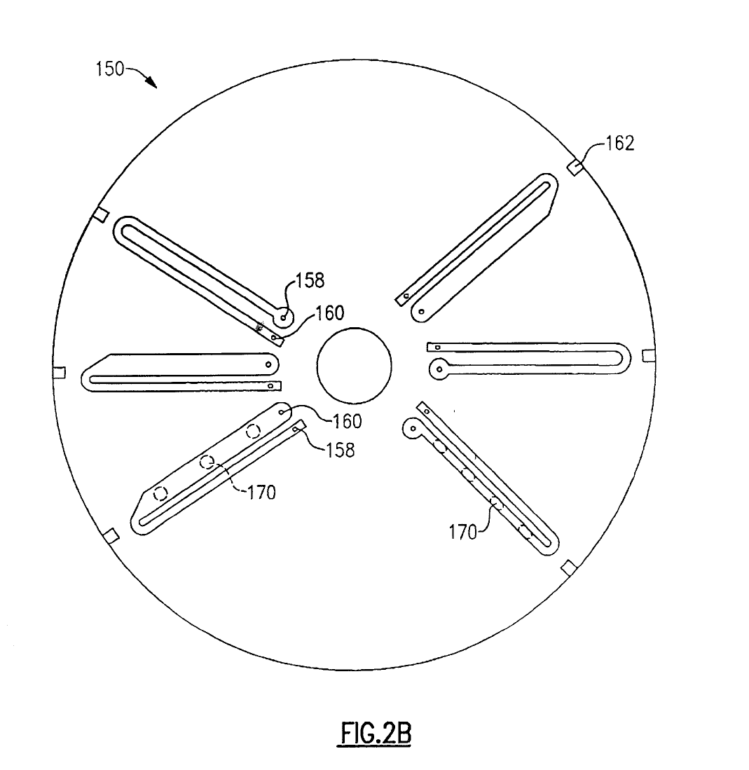

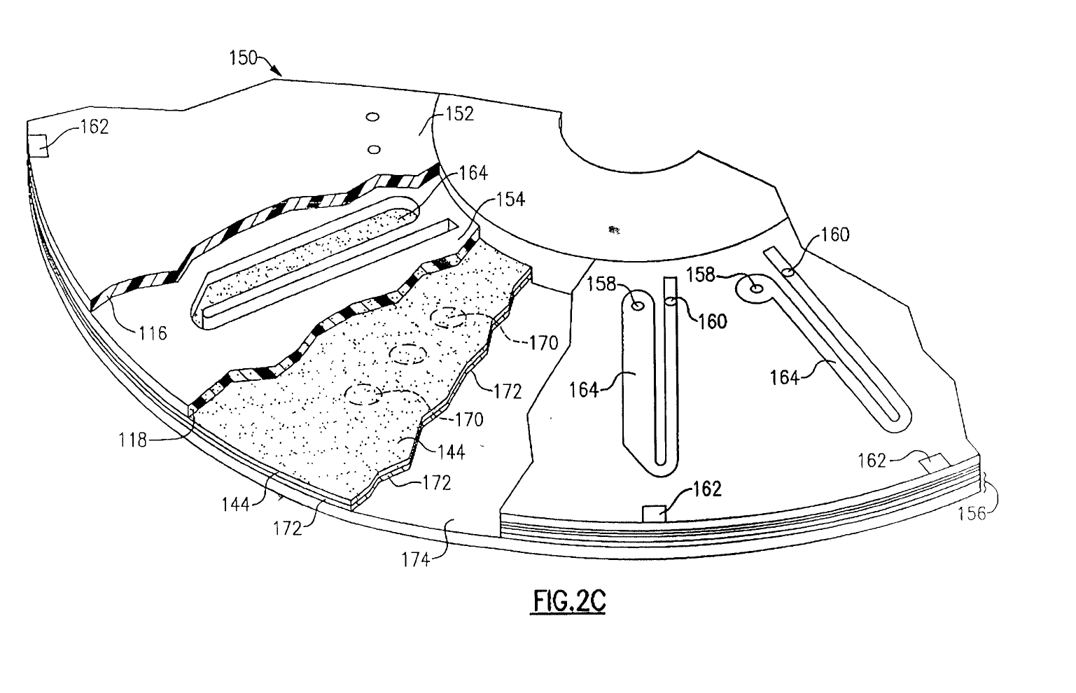

Referring to FIGS. 2A, 2B, and 2C, a transmissive optical disc 150 has a cap 152, a channel layer 154, and a substrate 156. Cap 152 includes inlet ports 158 and vent ports 160 and is preferably formed mainly from polycarbonate. Trigger marks 162 similar to those for disc 100 may be included. Channel layer 154 has fluidic circuits 164, which can have structure and use similar to those described in conjunction with FIGS. 1A, 1B, and 1C.

Substrate 156 may include target zones 170, and preferably includes polycarbonate layer 174. Substrate 156 may, but need not, have a thin semi-reflective layer 172 deposited on top of layer 174. Semi-reflective layer 172 is preferably significantly thinner than reflective layer 148 on substrate 156 of reflective disc 100 (FIGS. 1A-1C). Semi-reflective layer 172 is preferably formed form a metal, such as aluminum or gold, but is sufficiently thin to allow a portion of an incident light beam to penetrate and pass through layer 172, while some of the incident light is reflected back. A gold film layer, for example, is 95% reflective at a thickness greater than about 700 Å, while the transmission of light through the gold film is about 50% transmissive at approximately 100 Å.

FIG. 2C is a cut-away perspective view of disc 150. The semi-reflective nature of layer 172 makes its entire surface available for target zones, including virtual zones defined by trigger marks or specially encoded data patterns on the disc. Target zones 170 may also be formed by marking the designated area in the indicated shape or alternatively in any desired shape. Markings to indicate target zone 170 may be made on semi-reflective layer 172 or on a bottom portion of substrate 156 (under the disc). Target zones 170 may be created by silk screening ink onto semi-reflective layer 172.

An active capture layer 180 is applied over semi-reflective layer 172. Capture layer 180 may be formed from the same materials as described above in conjunction with layer 144 (FIG. 1C) and serves substantially the same purpose when a sample is provided through an opening in disc 150 and the disc is rotated. In transmissive disc 150, there is no reflective layer comparable to reflective layer 116 in reflective disc 100 (FIG. 1C).

Optical Disc Drive

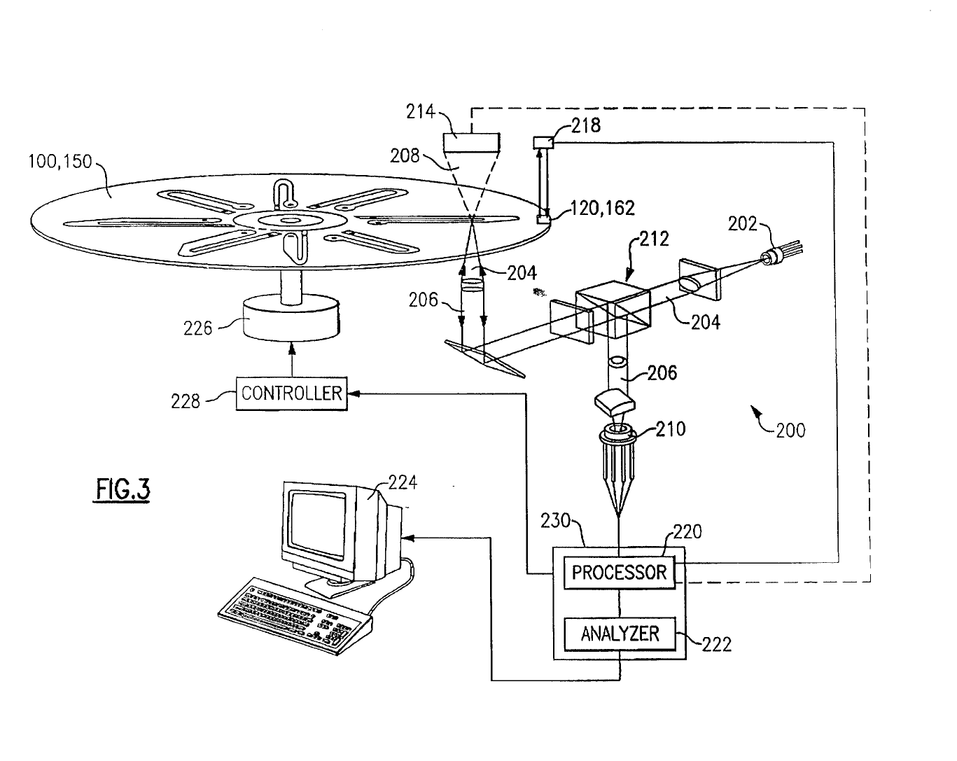

FIG. 3 shows an optical disc reader system 200. This system may be a conventional reader for CD, CD-R, DVD, or other known comparable format, a modified version of such a drive, or a completely distinct dedicated device. The basic components are a motor for rotating the disc, a light system for providing light, and a detection system for detecting light.

A light source 202 provides light to optical components 212 to produce an incident light beam 204, a return beam 206, and a transmitted beam 208. In the case of reflective disc 100, return beam 206 is reflected from either reflective surface 148 or 116. Return beam 206 is provided back to optical components 212, and then to a bottom detector 210. For transmissive disc 150, a transmitted beam 208 is detected by a top detector 214. Optical components 212 can include a lens, a beam splitter, and a quarter wave plate that changes the polarization of the light beam so that the beam splitter directs a reflected beam through the lens to focus the reflected beam onto the detector. An astigmatic element, such as a cylindrical lens, may be provided between the beam splitter and detector to introduce astigmatism in the reflected light beam.

Data from detector 210 and/or detector 214 is provided to a computer 230 including a processor 220 and an analyzer 222. An image or output results can then be provided to a monitor 224. Computer 230 can represent a desktop computer, programmable logic, or some other processing device, and also can include a connection (such as over the Internet) to other processing and/or storage devices. A drive motor 226 and a controller 228 are provided for controlling the rotation and direction of disc 100 or 150. Controller 228 and the computer with processor 220 can be in communication or can be the same computer. Methods and systems for reading such a disc are also shown in Gordon, U.S. Pat. No. 5,892,577, which is incorporated herein by reference.

A hardware trigger sensor 218 may be used with either a reflective or transmissive disc. Triggering sensor 218 provides a signal to computer 230 (or to some other electronics) to allow for the collection of data by processor 220 only when incident beam 204 is on a target zone. Alternatively, software read from a disc can be used to control data collection by processor 220 independent of any physical marks on the disc.

The substrate layer may be impressed with a spiral track that starts at an innermost readable portion of the disc and then spirals out to an outermost readable portion of the disc. In a non-recordable CD, this track is made up of a series of embossed pits with varying length, each typically having a depth of approximately one-quarter the wavelength of the light that is used to read the disc. The varying lengths and spacing between the pits encode the operational data. The spiral groove of a recordable CD-like disc has a detectable dye rather than pits. This is where the operation information, such as the rotation rate, is recorded. Depending on the test, assay, or investigational protocol, the rotation rate may be variable with intervening or consecutive periods of acceleration, constant speed, and deceleration. These periods may be closely controlled both as to speed and time of rotation to provide, for example, mixing, agitation, or separation of fluids and suspensions with agents, reagents, antibodies, or other materials.

Numerous designs and configurations of an optical pickup and associated electronics may be used in the context of the embodiments of the present invention. Further details and alternative designs for compact discs and readers are described in Compact Disc Technology, by Nakajima and Ogawa, IOS Press, Inc. (1992); The Compact Disc Handbook, Digital Audio and Compact Disc Technology, by Baert et al. (eds.), Books Britain (1995); and CD-Rom Professional's CD-Recordable Handbook: The Complete Guide to Practical Desktop CD, Starrett et al. (eds.), ISBN:0910965188 (1996); all of which are incorporated herein in their entirety by reference.

The disc drive assembly is thus employed to rotate the disc, read and process any encoded operational information stored on the disc, analyze the liquid, chemical, biological, or biochemical investigational features in an assay region of the disc, to write information to the disc either before or after the material in the assay zone is analyzed by the read beam of the drive or deliver the information via various possible interfaces, such as Ethernet to a user, database, or anywhere the information could be utilized.

Cell Detection

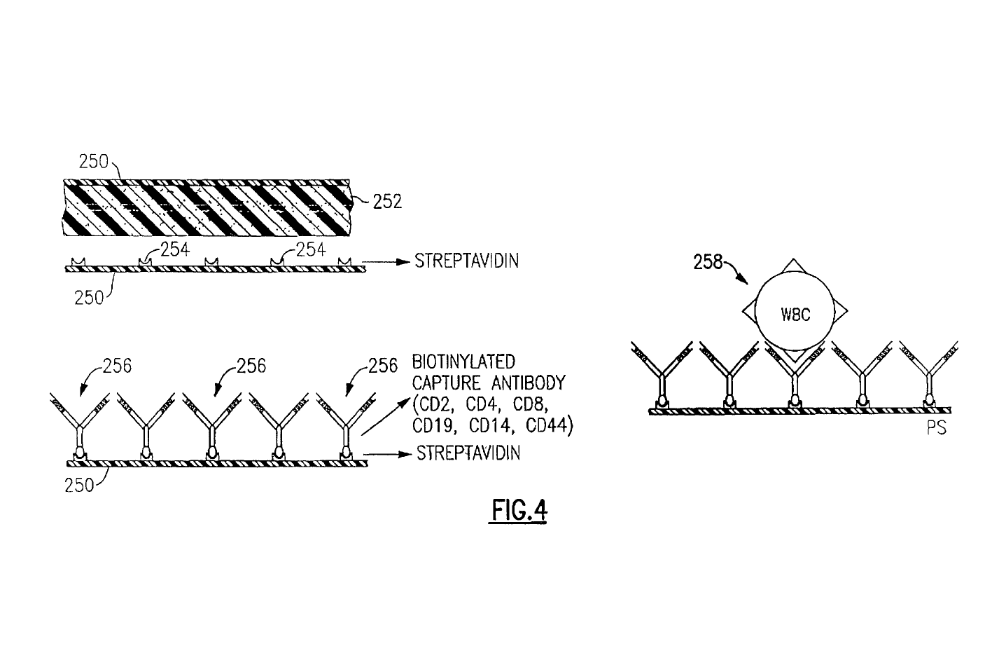

Referring to FIG. 4, in one embodiment of the present invention, a thick layer of polystyrene 250 is formed over a substrate 252 and is (optionally) layered with streptavidin 254. Cell capture antibodies 256 are attached to the strepavidin 254 through biotin. These antibodies can include biotinylated antibodies attached to Dextran-activated aldehyde coated over the streptavidin to create an ample number of binding sites for the capture antibody. This creates a strong capture chemistry that can specifically form robust bonds with white blood cells (WBCs) 258.

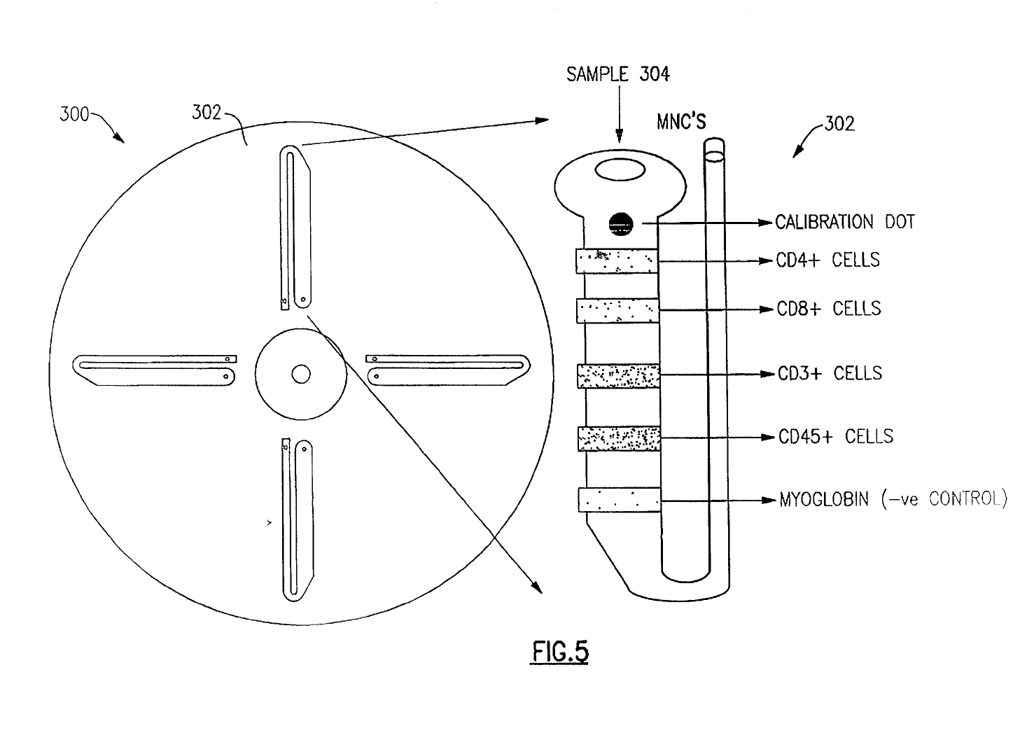

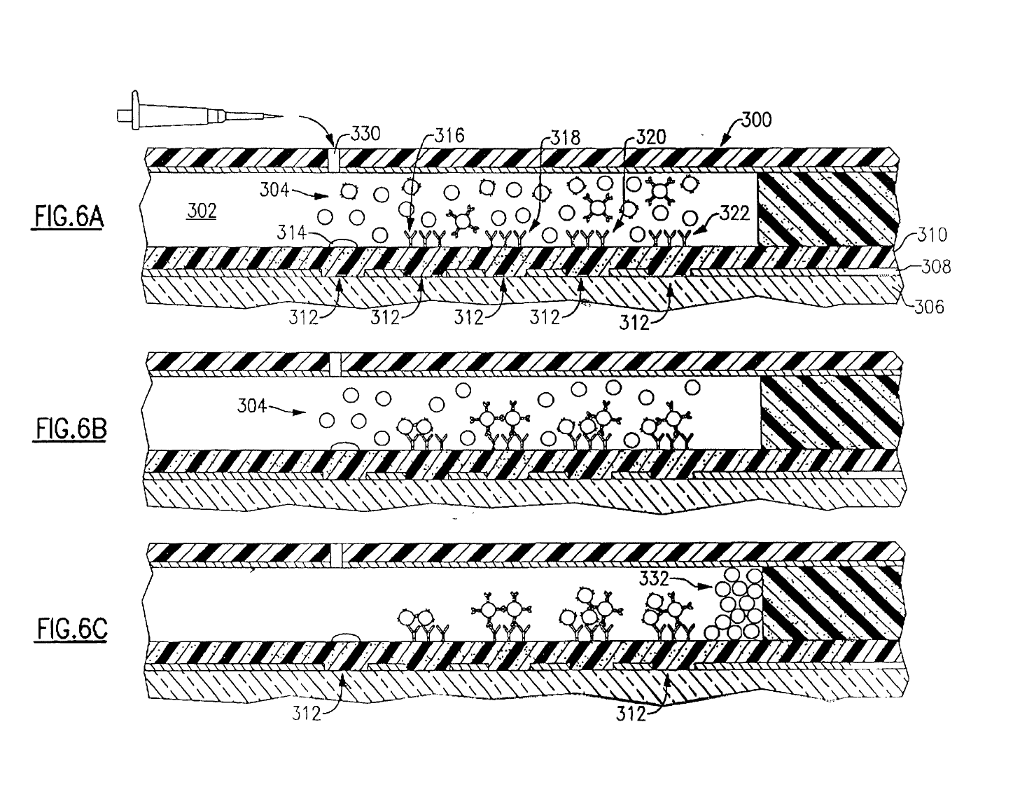

Referring to FIGS. 5 and 6A-6C, an optical disc 300 has a fluid circuit 302 for holding a sample. As shown in FIG. 6A, disc 300 has a light transparent substrate 306, a reflective layer 308, and a capture layer 310 internal to the disc. Portions of reflective layer 308 are removed (or openings were created when deposited) to produce viewing windows through which light can be directed at the locations of capture zones 312 where the antibodies are affixed. FIG. 6A shows five such capture zones with a calibration dot 314 over the first window and different antibodies 316, 318, 320, and 322 on capture layer 310 at locations over successive zones 312. Antibodies 316-322 may all be different, but may nonetheless be formed within the same fluidly coupled chamber. As indicated in FIG. 5 (which shows six captures zones including the calibration dot), these antibodies can include, for example, antibodies against CD4, CD8, CD3, CD45, and CD14. Other antibodies also could be used.

As shown in FIG. 6A, a sample is provided into the chamber through port 330, and centrifugal force from rotation of the disc causes sample 304 to move along the direction of capture zones 312. The cells in sample 304, as shown in FIG. 6B, have cell surface antigens that are bound by the antibodies. As indicated in FIG. 6C, the continued rotation causes unbound cells 332 to move to one end of the chamber away from capture zones 312, while in the capture zones, antibodies bind to antigens on cells. The rotation can be at one speed in one step, or in different steps at different rates (e.g., successively faster) to accomplish both moving the cells to the capture zones and moving the unbound cells away from the capture zones. Intermediate non-rotating steps may also be used.

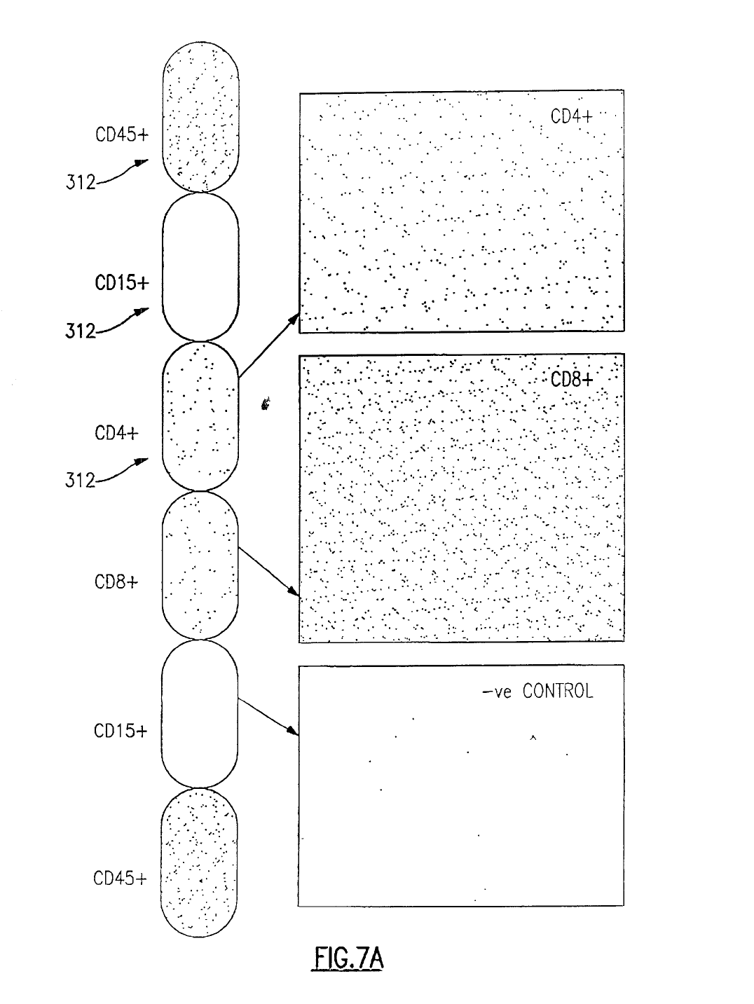

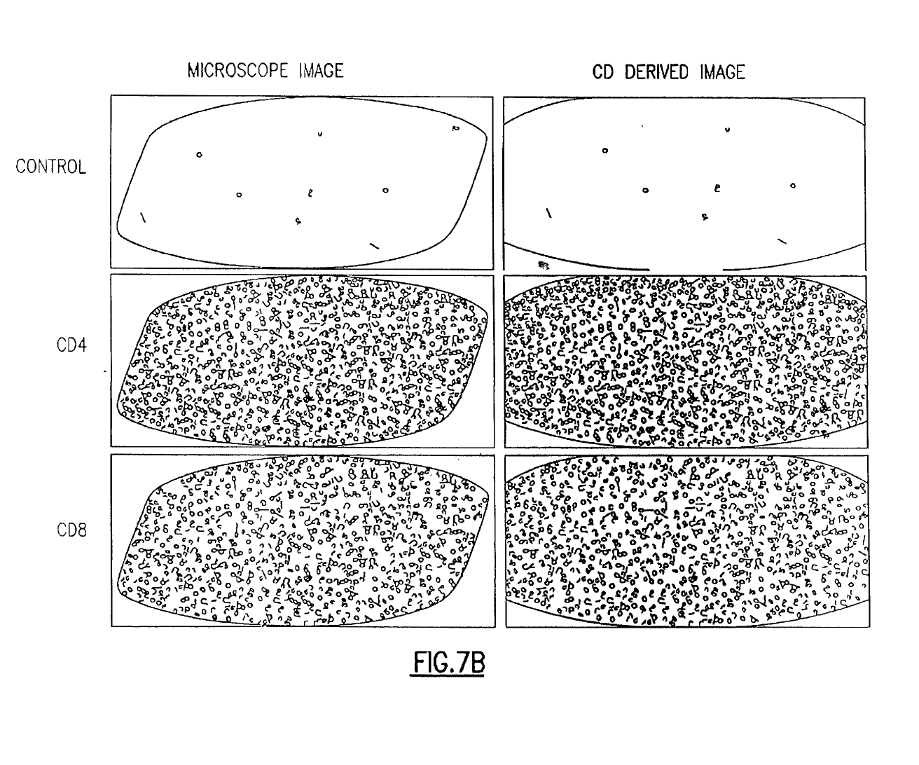

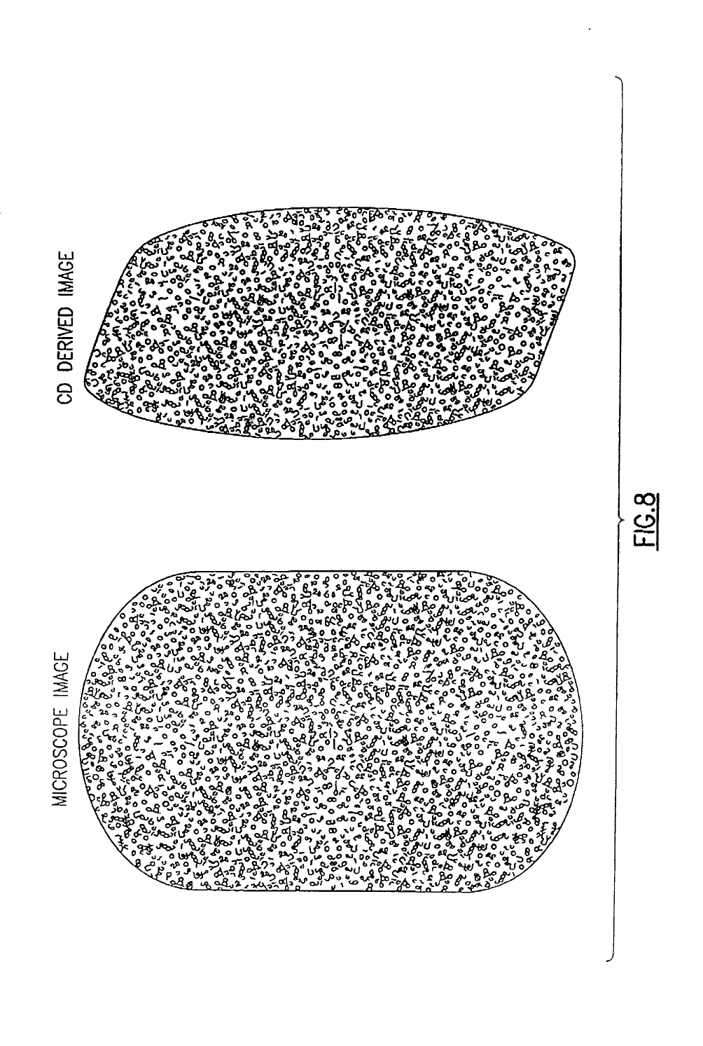

Referring also to FIG. 7A, an image obtained at a capture zone is shown for a series of cell surface antigens with enlarged views for CD4, CD8, and a control. As indicated here, the image is of a number of cells shown against a background field. FIG. 7B shows a close up view is shown with a comparison of a microscope image and a CD derived image for control, CD4, and CD8, and FIG. 8 shows another comparison of a microscope image and disc image. These images show that individual cells can be made visible against a background. Methods for detecting features are described in more detail in Provisional Application Serial Nos. 60/270,095, filed Feb. 2, 2001; and 60/292,108, filed May 18, 2001, each of which is incorporated herein by reference.

These cells can be detected through one of a variety of different methods including, for example, using edge detection hardware or software to detect and count sufficiently large changes in the level of transmitted or reflected light and thus count the transitions and hence the cells. Another method, described in more detail below, uses image (pattern) recognition software to identify cells against the background. Image recognition can distinguish WBCs from RBCs, and also distinguish neutrophils, monocytes, basophils, eosinophils, granulocytes, and lymphocytes.

An optical disc with tracks on the order of 1.6 microns apart can be used to image cells or aggregates on the disc. For example, a white blood cell would typically have a diameter of at least 5 and as many as 12 tracks, and therefore an image of that white blood cell can be obtained.

To obtain such an image, a transmissive disc of the type shown in FIGS. 2A-2C may be used (although a reflective disc would be operable), and to use a disc drive system of the type shown in FIG. 3 including trigger sensor 218 and top detector 214. Trigger detector 214 detects a trigger mark 162 in a transmissive disc and provides a signal to a computer that data is to be collected and/or processed when that mark is detected. As the light source passes across the tracks in the viewing window, an image is obtained for the received transmitted light. The top detector in this case can be a single detector, or an array of multiple detector elements oriented in the radial and/or circumferential direction. Such detectors and detection methods are described, for example, in Provisional Application Serial Nos. 60/247,465, filed Nov. 9, 2000; and 60/293,093, filed May 22, 2001, each of which is incorporated herein by reference.

After images such as those in FIGS. 7A, 7B, and 8 are obtained, the image data can be processed further with image recognition software designed to identify desired features. It is further desirable that the image recognition software not only have the ability to distinguish cells from background, but also one type of cell from another.

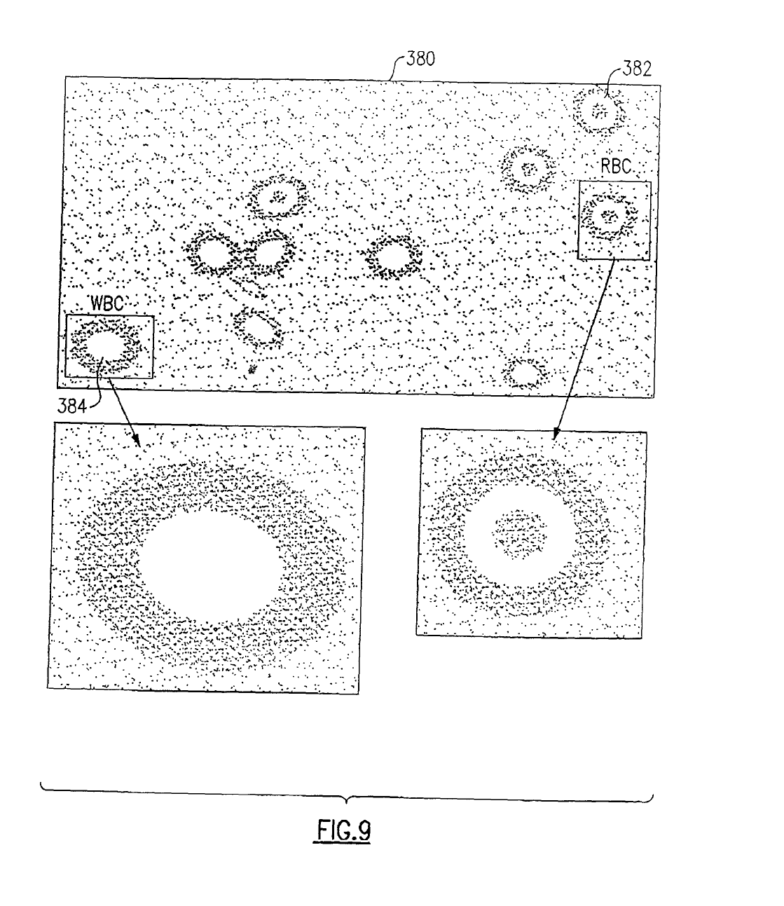

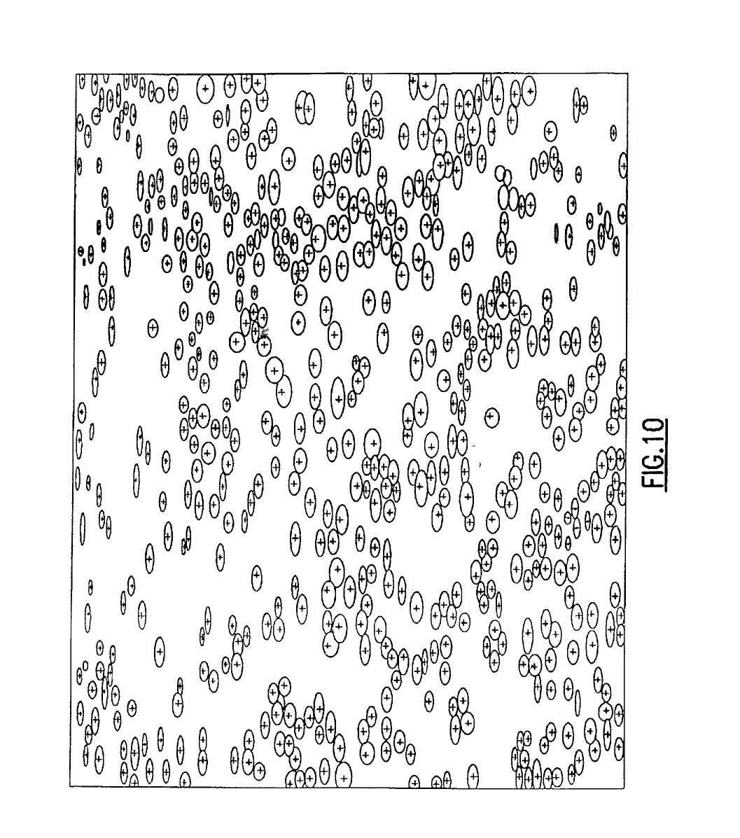

FIG. 9 shows an image 380 that includes both red blood cells 382 and white blood cells 384. As indicated in the enlarged views, these white and red cells have clearly distinct characteristics and thus can be detected against the background and can also be distinguished from each other with image recognition. In addition, it is also possible to distinguish types of white blood cells from each other, including lymphocytes, monocytes, neutrophils, eosinophils, granulocytes, and basophils.

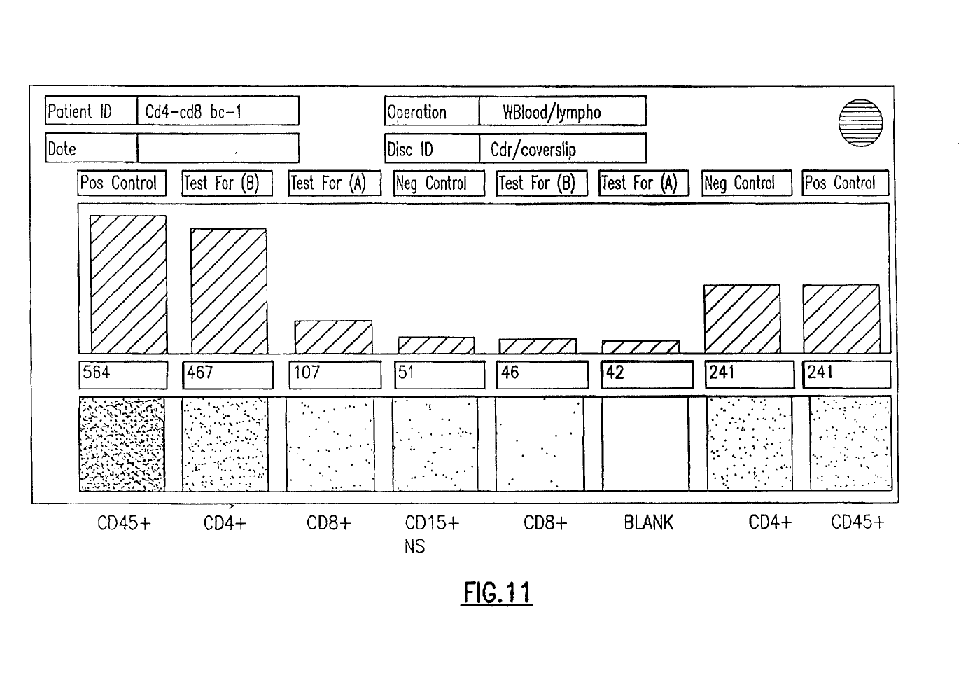

FIG. 10 shows a sample field with a number of cells with a plus sign indicating each object that is identified as a cell.

After the number of cells have been detected for every zone, the data can be displayed, e.g., in a single screen that provides an easy to view representation, such as that shown in FIG. 11, which indicates the specific counts and also has a bar graph to demonstrate relative numbers of cells. In the case of CD4/CD8, the system can also produce a ratio and any other mathematical calculations and comparisons that are desired.

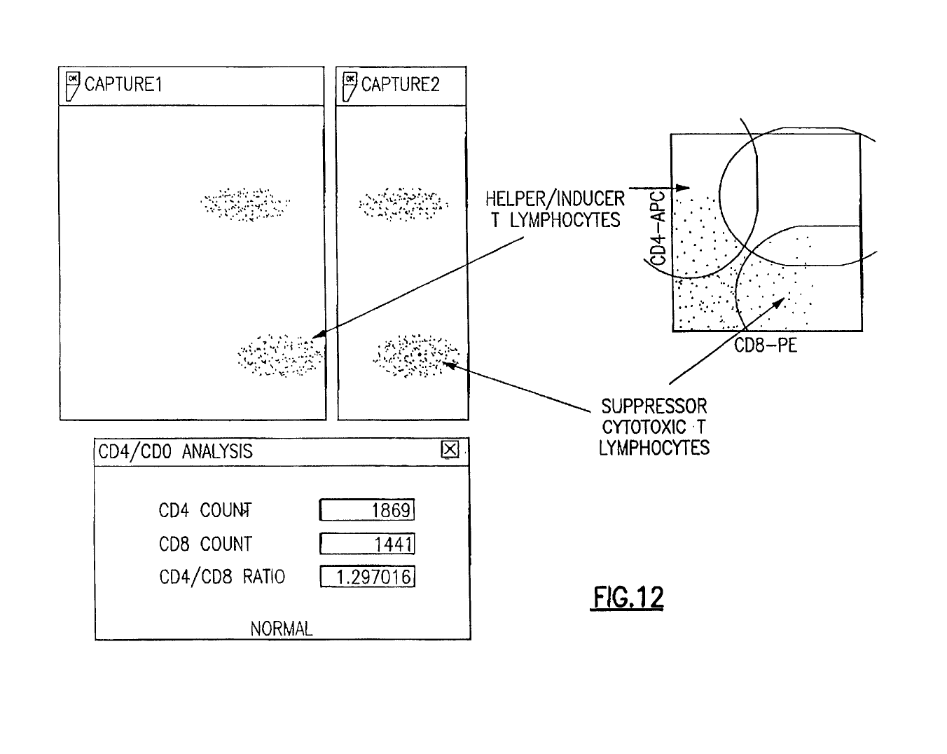

FIG. 12 provides a different view of the process showing cells in an image field being converted to a CD4 count, a CD8 count, and a ratio, with the output indicating that the ratio is in a normal range.

The following discussion is a more detailed example showing a particular embodiment of the present invention.

### EXAMPLE

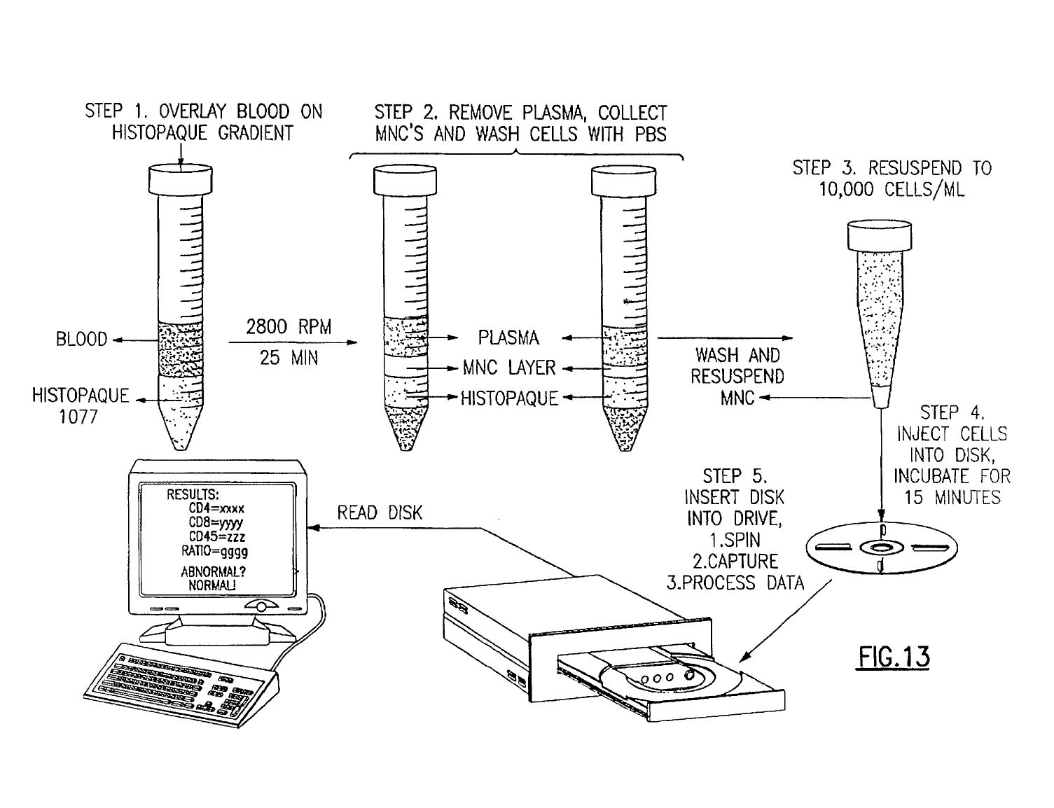

FIG. 13 illustrates a pictorial flow chart showing the creation of a sample, e.g., a sample of cells, the injection of that sample into a disc as shown in more detail in FIGS. 6A-6C, the use of a disc, and the provision of results as shown in FIGS. 11 and 12. The sum of the times and rotation rates and other details may be slightly different from the example as described below, but the basic steps would be similar nonetheless.

In a more detailed exemplary method, a reflective disc or transmissive disc substrate (106 in FIG. 1A; 156 in FIG. 2A) is cleaned using an air gun to remove any dust particles. The disc is rinsed twice with iso-propanol, using a spin coater. A 2% polystyrene is spin coated on the disc to give a very thick coating throughout.

The chemistry is then deposited. One embodiment includes a three step deposition protocol that incubates: streptavidin, incubated for 30 minutes; biotinylated first antibody incubated for 60 minutes; and a second capture antibody incubated for 30 minutes. The first antibody can be raised in a first species (e.g., sheep) against a type of immunoglobulin (e.g., IgG, IgE, IgM) of a second species (e.g., mouse). The second capture antibody is raised in the second species against a specific cell surface antigen (e.g., CD4, CD8). The steps are done at room temperature in a humidity chamber using washing and drying steps between depositions.

Briefly, 1 μl of 1 mg/ml streptavidin in phosphate buffered saline is layered over each window and incubated for 30 minutes. Excess streptavidin is rinsed off using distilled water and the disc is dried. Equal volumes of biotinylated anti-mouse IgG (125 μg/ml in PBS) and activated dextran aldehyde (200 μg/ml) are combined. Dextran aldehyde (DCHO)-biotinylated anti-mouse IgG is layered over streptavidin in each capture window and incubated for 60 minutes or overnight in refrigerator. Excess reagent is rinsed and the disc is spun dry.

As shown in FIG. 5, there can be a number of radially oriented viewing windows with different tests, such as CD4 (window 2), CD8 (window 3), CD3 (window 4), and CD45 (window 5), and negative control (window 6), using mouse IgG antibodies against these human cell surface antigens. This prepared substrate is incubated for 30 minutes or overnight in the refrigerator.

The pattern of chemistry deposition is thus as follows:

| Window | 1 2 | 3 4 | 5 6 | 7 8 |
|---|---|---|---|---|
| 1^{st} Layer | Streptavidin | Streptavidin | Streptavidin | Streptavidin |
| 1^{st} Antibodies | B-anti Mouse | B-anti Mouse | B-anti Mouse | B-anti Mouse |
| | IgG + DCHO | IgG + DCHO | IgG + DCHO | IgG + DCHO |
| 2^{nd} Antibodies | Mouse Anti- | Mouse Anti- | Mouse Anti- | Mouse Arti |
| | human CD4 | human CD8 | human CD3 | human CD45 |

The disc is assembled using an adhesive layer that may, for example, be 25, 50, or 100 microns thick (104 and 154 in FIGS. 1A and 2A), with a stamped out portion, such as a U-shape, to create a fluidic channel, and a clear cap 152 (FIG. 2A, for use with a transmissive disc with a top detector) or a cap 102 with a reflective layer 148 located over the capture zones (FIG. 1A, for use with a reflective disc with a bottom detector).

Because blood is being analyzed, the disc can be leak checked first to make sure none of the chambers leak during spinning of the disc with the sample in situ. Each channel is filled with a blocking agent, StabilGuard, such as PBS-Tween. The block is for at least 1 hour. The disc is spun at 5000 rpm for 5 minutes to leak proof and check disc stability. After checking for leaks. the disc is placed in a vacuum chamber for 24 hours. After vacuuming for 24 hours, discs are placed in a vacuum pouch and stored in refrigerator until use.

The following method is for processing the samples, steps of which are also shown in FIG. 13. Mononuclear cells (MNC) are purified by a density gradient centrifugation method, e.g., using a Becton Dickinson CPT Vacutainer or Histopaque 1077. Blood (4-8 ml) is collected directly into a 4 or 8 ml EDTA containing CPT Vacutainer. The tubes are centrifuged at 1500 to 1800 RCFs (2800 rpm) in a biohazard centrifuge with horizontal rotor and swing out buckets for 25 minutes at room temperature. The blood is preferably used within two hours of collection. After centrifugation, plasma overlying the mononuclear cell fraction is removed, leaving about 2 mm of plasma above an MNC layer. MNC are collected and washed with PBS. Cells are pelleted by centrifuge at 300 RCFs (1200 rpm) for 10 minutes at room temperature. The supernatant is removed and the pellet containing the MNC is resuspended in PBS by tapping the tube gently. Two more washes are done at 178 RCFs (950 rpm) for 10 minutes each at room temperature to remove platelets. The final pellet is resuspended to a cell count of 30,000 cells/μl. If a blood sample cannot be processed immediately, mononuclear cells after the first centrifugation can be resuspended in plasma by gently inverting the CPT tube several times and stored for up to 24 hours at room temperature. Within 24 hours, the cells in the plasma can be collected and washed as described above.

MNC cells (7 μl of in PBS) are injected into the disc chamber, and inlet and outlet ports of the chamber are sealed with tape. The disc is incubated for 30 minutes at room temperature, and then scanned using a 780 nm laser in an optical drive with a top detector to image the capture field as described above.

Software is preferably encoded on the disc to instruct the drive to automatically perform the following acts: (a) centrifuge the disc to spin off excess unbound cells in one or more stages, (b) image specific capture windows, and (c) processing data, including counting the specifically-captured cells in each capture zone and deriving the ratio of CD4/CD8 (or which ever ratio is programmed to be determined).

During the processing step, the software reads across each capture zone image and marks cells as it encounters them. For example, following estimation of number of CD4+ and CD8+ cells, the software calculates the ratio of CD4+/CD8+ cells and displays both the absolute numbers of cells in CD4+, CD8+, CD3+ and CD45+ capture zones per microliter of whole blood and also the CD4+/CD8+ ratio. The entire process takes about 12 minutes from inserting the disc into the optical drive to obtaining the numbers and ratios.

In one embodiment, the disc is a forward Wobble Set FDL21:13707 or FDL21:1270 CD-R disc coated with 300 nm of gold as the encoded information layer. On a reflective disc, viewing windows of size 2×1 mm oval are etched out of the reflective layer by known lithography techniques. In some designs of transmissive disc, no separate viewing windows are etched, and the entire disc is available for use. The adhesive layer is Fraylock adhesive DBL 201 Rev C 3M94661. The cover is a clear disc with 48 sample inlets with a diameter of 0.040 inches located equidistantly at radius 26 mm. The data disc is scanned and read with the software at speed 4× and sample rate 2.67 MHz using CD4/CD8 counting software.

This assay is a generic homogeneous solid phase cell capture assay for the rapid determination of absolute number of CD4+ and CD8+ T-lymphocyte populations and ratio of CD4+/CD8+ lymphocytes in blood samples. The test, which is run within a small chamber incorporated into a CD-ROM, determines the number of CD4+, CD8+, CD2+, CD3+ and CD45+ cells captured by the specific antibodies on the capture zones in 7 μl of mononuclear cells (MNC) isolated from whole blood. The test is based upon the principle of localized cell capture on specific locations on the disc. Several specific cell capture zones are created on the disc by localized application of capture chemistries based upon monoclonal or polyclonal antibodies to particular blood cell surface antigens. Upon flooding the chamber with the MNC blood (30,000 cells/μl), cells expressing antigens CD4, CD8, CD2, CD3 and CD45 are captured in the capture zones on the disc. Also incorporated within the bar code are defined negative control areas.

Blood (4 ml) is either drawn into a CPT tube or blood already drawn is overlay over a CPT Vacutainer. The tube is spun at 1200 g for 25 min at 25° C. The pure mononuclear fraction at the interface of the gel and the blood is collected, the pellet is washed twice with PBS, the washed cells are centrifuged at 1200 rpm for 5 min, and the pellet is resuspended to give a cell concentration of 30,000 cells/μl. 7 μl of the MNC is applied to the chamber, incubated for 30 minutes at room temperature with the disc stationary. The channels are sealed with tape. The disc is scanned and read with the software at speed 4× (i.e., 1600 rpms) and sample rate 2.67 MHz.

Reagents Used in the Example

Streptavidin (Sigma, cat. #S-4762): Add de-ionized water to make a 5 mg/ml solution, aliquot and store at −30° C. To use, add Tris buffer for a final concentration of 1 mg/ml.

Positive control: CD45 (Sigma, Lot #038H4892, cat #C7556). Store at 2-8° C.

First capture antibody: Biotinylated anti-mouse IgG (raised in sheep, Vector laboratories, lot #L0602, Catalog #BA-9200) Stock solution 1.5 mg/ml made in distilled water. Working b-IgG solution 125 μg/ml in 0.1M PBS. Store at 2-8° C. May be kept at −30° C. for long term storage.

Aldehyde activated Dextran (Pierce, lot #97111761, cat #1856167). Stock solution stock solution 5 mg/ml in PBS, store at 2-8° C.

Second capture antibody: CD4 (DAKO, cat #M0716) CD8 (DAKO, cat #M0707), CD2 (DAKO, cat #M720), CD45 (DAKO, cat #M0701), CD14 (DAKO, cat #M825), and CD3 (DAKO, cat #M7193). Store at 2-8° C.

Negative control: CD14 (Sigma, Lot #128H4867, cat #C7556). Store at 2-8° C.

Phosphate Buffered Saline (PBS), pH 7.4 (Life Technologies/GIBCO BRL, cat. #10010-023) or equivalent. Store at room temperature Isopropyl alcohol, 90-100%

RBC Lysing Protocol

Ammonium Chloride Lysing Buffer:

A 10×stock of ammonium chloride lysing buffer should be stored at 2 to 8° C.

8.29 grams NH₄Cl

1.09 grams KHCO₃

37.0 mgs disodium EDTA

QS to 100 mls with glass-distilled water

1×solution=dilute 10 mls of 10× to 100 mls with 1×PBS. Filter. The pH of the solution should be 7.3 to 7.4. Store at room temperature. Use within one week.

Procedure:

1. For every 100 μl of blood add 2 ml of lysing buffer. (It is preferable to do this procedure in a biohazard hood.)

2. Vortex and incubate for 10 minutes at room temperature

3. Centrifuge the blood at 1200 rpm for 5 minutes at room temperature, using the centrifuge in the biohazard hood.

4. Remove supernatant and wash cells with PBS. Centrifuge cells.

5. Repeat wash one more time.

6. Calculate the total amount of WBCs and make the final concentration of WBCs 30,000 cells/μl for sample injection.

While this invention has been described in detail with reference to certain preferred embodiments, it should be appreciated that the present invention is not limited to those precise embodiments. For example, software can be used to identify a viewing window rather than using a trigger mark and a trigger detector.

What is claimed is:

1. A method of conducting an assay with an optical disc and disc drive, the method comprising:
providing a sample of cells on a disc surface in a chamber in a disc, the chamber including at least one capture zone with a capture agent;
loading the disc into an optical reader;
rotating the optical disc;
directing an incident beam of electromagnetic radiation to the capture zone;
detecting a beam of electromagnetic radiation formed after interacting with the disc at the capture zone;
converting the detected beam into an output signal; and
analyzing the output signal to extract therefrom information relating to the number of cells captured at the capture zone.

2. The method according to claim 1, wherein the chamber with the disc surface supporting the sample is internal to the disc and is bounded on opposite sides by a substrate and cap.

3. The method according to claim 1, wherein the optical disc is constructed with a reflective layer such that light directed to the capture zone and not striking a cell is reflected.

4. The method according to claim 1, wherein the optical disc is constructed such that light directed to the capture zone and not striking a cell is transmitted through the optical disc, the disc being between the light source and the detector.

5. The method according to any one of claim 1, wherein the disc surface is coated with a first group of cell capture agents.

6. The method according to claim 5, wherein the cell capture agents define a discrete capture zone.

7. The method according to claim 6, wherein a second group of cell capture agents define a second discrete capture zones in a predetermined pattern.

8. The method according to claim 7, wherein the first and second captures zones are in one chamber.

9. The method according to claim 5, wherein the cell capture agents are for binding with cell surface antigen.

10. The method according to claim 9, wherein the cell surface antigen is selected from the CD family of antigens.

11. The method according to claim 10, wherein the cell surface antigen is selected from the group consisting of CD3, CD4, CD8, and CD45.

12. The method according to claim 1, further including:
directing the sample of cells into proximity with the cell capture agents;
incubating the cells in the presence of the capture agents; and
allowing the cells to specifically bind to the capture agents.

13. The method according to claim 12, further including analyzing the number of cells captured to thereby determine a cell concentration in the sample.

14. The method of claim 13, wherein the analyzing includes detecting sufficiently large changes in the level of light reflected from or transmitted through the disc.

15. The method of claim 13, wherein the analyzing includes using image recognition to count captured cells.

16. The method of claim 15, wherein the image recognition distinguished one type of white blood cell from another.

17. The method of claim 1, wherein the chamber has a plurality of capture zones, each having a different cell capture agent.

18. The method of claim 17, wherein the rotating includes rotating for a sufficient period of time at a sufficient speed so that the cells have an opportunity to bind with the capture molecules.

19. The method of claim 18, wherein the rotating includes rotating for a sufficient period of time at a sufficient speed so that unbound cells are moved away from the capture zones.

20. The method of claim 19, wherein the rotating is done at a single speed.

21. The method of claim 17, further comprising counting the captured cells in each of the capture zones and providing an output including the counts.

22. The method of claim 21, wherein the output includes counts for CD4 cells and CD8 cells, and a ratio of CD4 to CD8 cells.

23. An optical disc comprising:
a substrate;
a cap parallel to the substrate, a chamber defined therebetween and including capture zones; and
a capture layer over the substrate at the capture zones, such that a first capture zone has first cell capture agents and a second capture zone has a second cell capture agents.

24. The disc of claim 23, wherein the agents are antibodies for cell surface antigens on white blood cells.

25. The disc of claim 24, wherein the agents are antibodies for CD4 and CD8.

26. An optical disc and drive system for receiving a sample, the system comprising:
a disc including:
a substrate;
a cap parallel to the substrate, a chamber defined therebetween and including capture zones;
a capture layer over the substrate at the capture zones, such that a first capture zone has first cell capture agents and a second capture zone has a second cell capture agents;
a light source for directing light to the disc at the capture zones;
a detector for detecting light reflected from or transmitted through the disc at the capture zones and providing a signal; and
a processor for using the signal to count items in the sample bound to the capture molecules.

27. The disc of claim 26, wherein the detector is on the same side of the disc as the light source for detecting light reflected from the captures zones.

28. The disc of claim 26, wherein the detector is on the opposite side of the disc as the light source for detecting light transmitted through the capture zones.

29. The disc of claim 26, wherein the processor includes image recognition software for detecting imaged cells.
